(19) United States
(12) Patent Application Publication
(10) Pub. No.: US 2011/0088451 A1
(43) Pub. Date: Apr. 21, 2011

(54) NONVISUAL INDICATION OF AN UNWANTED CHEMICAL IN AN INGESTIBLE SUBSTANCE

(76) Inventor: Stephen F. Holmes, Portland, OR (US)

(21) Appl. No.: 12/907,969

(22) Filed: Oct. 19, 2010

Related U.S. Application Data
(60) Provisional application No. 61/253,048, filed on Oct. 19, 2009.

(51) Int. Cl. G01N 33/02; G01N 33/00

(52) U.S. Cl. 73/23.34; 73/865.8; 73/865.7

(57) ABSTRACT

Methods and systems for detecting and indicating the presence of undesirable substances in ingestible substance such as food or beverages. More specifically, systems and methods for nonvisually and concealably indicating and detecting the presence of unwanted or undesired drugs in beverages and/or food.

### CROSS-REFERENCE TO RELATED APPLICATIONS

This application claims the benefit under 35 U.S.C. §119(e) of U.S. Provisional Patent Application Ser. No. 61/253,048, filed Oct. 19, 2009 and entitled “Methods and Systems for Performing a Concealed Test to Determine the Presence of an Unwanted Drug in a Beverage”, the disclosure of which is herein incorporated by reference.

### BACKGROUND

The present invention relates to methods and systems for detecting and indicating the presence of undesirable substances in ingestible substance such as food or beverages. More specifically, the invention relates to systems and methods for nonvisually and concealably indicating and detecting the presence of unwanted or undesired drugs in a beverage or food.

Certain drugs, sometimes referred to as “date rape drugs,” have been used to facilitate sexual assaults on unsuspecting victims. Typically the drug is surreptitiously placed in the intended victim's beverage or food. This is a relatively easy process because this class of drug typically provides little or no indication of its presence, creating little or no color change, detectable smell, or change in flavor in the drugged substance that could detected by the intended victim. Once consumed, the drugs typically used for this purpose can create weakness, confusion, and/or unconsciousness in the victim, who typically has no memory of what transpired while they were drugged. Some of these drugs, when combined with alcohol consumption, may exhibit an even more pronounced sedating effect.

While the victim is unconscious or disoriented, they may be subjected to sexual assault, theft, or other abuses by the person who drugged them.

Some pharmaceuticals which have been used as date rape drugs include flunitrazepam (trade name ROHYPNOL, and street name “roofie”), gamma-hydroxybutyric acid (or GHB), and ketamine. Similar acting drugs may also be used as date rape drugs, such as clonazepam (trade name KLONOPIN in the U.S. and RIVOTRIL in Mexico), alprazolam (trade name XANAX), and other benzodiazepines including temazepam (trade name RESTORIL) and midazolam.

Currently available tests for the detection of such drugs focus on creating a visible indicator that that the drug is present. Selected testing methods and systems are described in (a) U.S. Pat. Nos. 4,992,296; 5,457,054; 6,153,147; 6,703,216; 6,713,306; and 7,238,533; (b) U.S. Patent Application Nos. US2001/0046710; US2003/0026731; US2003/0224474; US2004/0146429; US2007/0065338; US2007/0099300; US2008/0006600; US2008/0102482; and US2009/0196675; (c) PCT Patent Application No. WO2005/059541; and (d) U.K. Patent No. 2436362, all of which are incorporated herein by reference.

The above types of tests are typical, in that they involve a tester device that, when exposed to a liquid, exhibits a visual color change when a target drug is present in the liquid. See for example U.S. Pat. No. 7,238,533, which discloses a color change in applied finger nail polish; U.S. Patent Application Publication No. 2004/0146429, which discloses an embedded so-called colorimetric indicator in a porous material used as a cocktail napkin, coaster, placemat, menu, matchbook, drink carrier, flyer, coupon, personal test kit or business card; and U.S. Patent Application Publication No. 2007/0099300, which discloses a color change on a straw. These tests, however, may not be readily concealable, as each test requires a visual analysis in order to determine the results of the test. Opportunities for testing may therefore be limited in a social setting and in the presence of the person who is suspected of placing an unwanted drug in a beverage.

What is needed is a test system that can verify the presence or absence of a target drug in a food or beverage without requiring a visual evaluation, so that the person suspected of tampering with the food or beverage need never know that they are under suspicion.

### BRIEF SUMMARY

In one embodiment, the invention includes a non-visual verification method that includes selecting an ingestible substance, using a detector on the selected, ingestible substance, and non-visually indicating whether a target substance is present in the ingestible substance.

In another embodiment, the invention includes a non-visual verification method that includes selecting an ingestible substance; making a detector for non-visual display of whether a target substance is present in the selected, ingestible substance; operating the detector and non-visually indicating whether a target substance is present in the ingestible substance.

In yet another embodiment, the invention is characterized as a non-visual verification system, where the non-visual verification system includes a non-visual, sensing mechanism constructed to verify whether a target drug is present in an ingestible substance by producing a non-visual indicator that can be understood by a user.

In yet another embodiment, the invention is characterized as a non-visual verification device, where the non-visual verification device includes a non-visual, sensing component constructed to verify whether a target drug is present in an ingestible substance by producing a non-visual indicator that can be understood by a user.

### BRIEF DESCRIPTION OF THE SEVERAL VIEWS OF THE DRAWINGS

FIG. 1 is a flowchart depicting a non-visual verification method, according to a selected embodiment of the invention.

FIG. 2 is a flowchart depicting a non-visual verification method, according to another selected embodiment of the invention.

FIG. 3 is a schematic depiction of a non-visual verification system, according to yet another selected embodiment of the invention.

### DETAILED DESCRIPTION

The present invention includes systems and methods for performing a non-visual test to determine the presence of a target substance in an ingestible substance. In particular, the invention includes systems and methods for performing a non-visual test to determine the presence of a target substance in an ingestible substance, where the target substance may be indicated both non-visually and concealably.

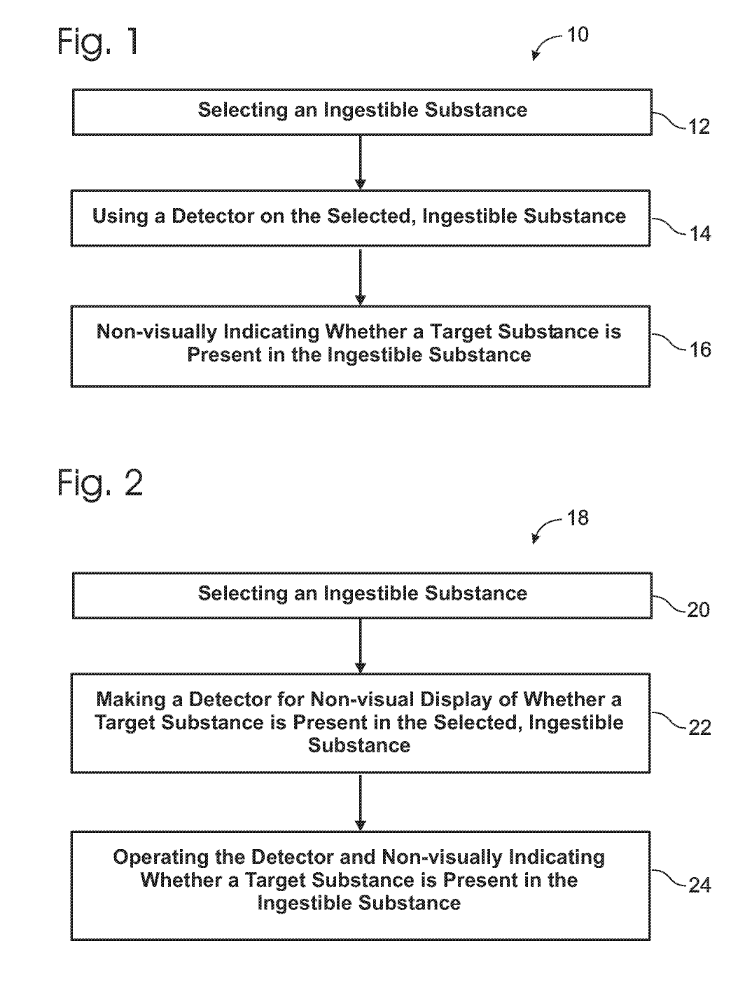

One embodiment of the present method is depicted as flowchart 10 in FIG. 1. The method includes selecting an ingestible substance, at 12; using a detector on the selected, ingestible substance, at 14; and non-visually indicating whether a target substance is present in the ingestible substance, at 16.

A second embodiment of the present method is depicted as flowchart 18 in FIG. 2. The method of FIG. 2 includes selecting an ingestible substance, at 20; making a detector for non-visual display of whether a target substance is present in the selected, ingestible substance, at 22; and operating the detector and non-visually indicating whether a target substance is present in the ingestible substance, at 24.

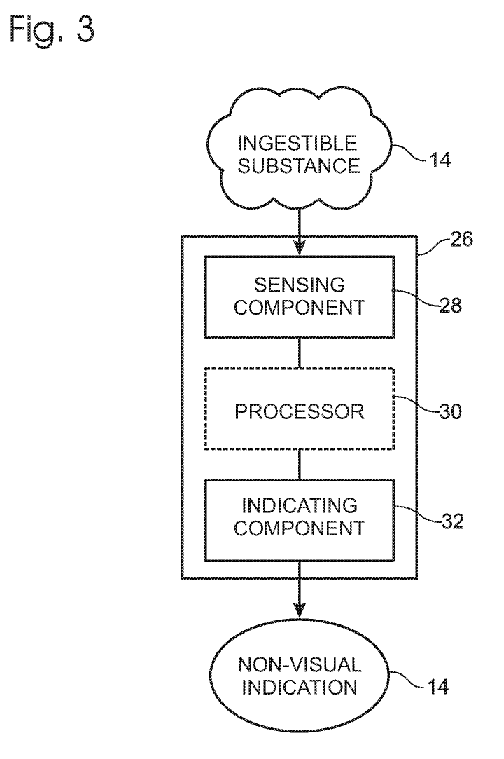

The disclosed methods may be performed using a non-visual verification system 26, as depicted schematically in FIG. 3. The non-visual verification system includes a sensing component 28, and an indicating component at 30. The non-visual verification system optionally further includes a processor 30, as will be discussed below.

The non-visual verification system may be used to test an ingestible substance, prior to consumption, for one or more specific target substances. That target substance(s) is typically an unwanted or undesired substance, such as a drug or a toxin. More particularly, the target substance may be a drug intended to confuse or sedate the consumer. Selected embodiments of the target substance include flunitrazepam, gamma-hydroxybutyric, ketamine, clonazepam, alprazolam, temazepam, and midazolam, without limitation.

The non-visual verification system is typically configured so that the ingestible substance may be tested for the target substance concealably. As used herein, the test is administered concealably where the ingestible substance may be tested for the target substance without alerting bystanders or companions that the test has been administered, or revealing the results of the test.

The non-visual verification system includes a sensing component, where the selected ingestible substance interacts with the system. The sensing component may be as simple as a molecule or a molecular array that incorporates one or more binding sites that are complementary to the target substance, such that binding the target substance results directly or indirectly in a non-visual response.

The ingestible substance to be tested may be any solid, semi-solid, or liquid that is intended for ingestion. The ingestible substance may also be a food or beverage. In one aspect of the invention, the ingestible substance is a liquid or semi-liquid, to facilitate detection of the target substance by chemical or physical interaction. The ingestible substance may also include chewing gum, toothpaste, and other substances that may be placed in the mouth, but not be intended to be swallowed.

The non-visual verification system typically incorporates an indicating component capable of indicating the presence of the selected target substance via a non-visual indication. In one embodiment, the indicating component is directly coupled to the sensing component, such as via molecular recognition, where binding the target substance triggers a molecular modification that produces that desired non-visual indication, such as via a change in flavor, odor, or texture.

In one particular embodiment, the non-visual verification system may correspond to an indicator substance that is selectively reactive toward the target substance (the sensing component), and produces an identifiable flavor compound in the presence of the target substance (the indicating component). Preferably, such substances react with the desired target substance with both selectivity and specificity, so as to avoid false positives. For example, the interaction between an indicator substance and the target substance may be similar to that of specific binding pairs. The indicator substance may be designed so that binding the target substance triggers a molecular rearrangement of the indicator substance, creating a distinct flavor change. Alternatively, or in addition, binding the target substance may trigger the release of one or more small molecules that are then detectable by flavor or odor.

Such an indicator substance may be utilized by placement in the users mouth, or by placing a component of the non-visual verification system that includes the indicator substance in the users mouth. Selected embodiments of the indicator substance may produce a bitter, or otherwise identifiable, taste to the user. The indicator substance may include more than one constituent element, and may correspond to any composition, element, material, reagent, or solution, or combination thereof, that is suitable for producing such an identifiable or bitter taste upon reaction with the target substance. In one embodiment, the non-visual verification system incorporates multiple indicator substances, each configured to react selectively with one or more target substances to produce a range of individually identifiable flavors. Such a multi-target system may be useful for the detection of multiple targets of interest simultaneously.

Where the indicating component is intended to produce a non-visual indication that is a flavor, the indicating component may be present on a strip of paper, a dissolvable strip of material, a saliva-dissolvable material, or a chewing gum-type of material, which a user may put in their mouth prior to ingesting the ingestible substance. The substance may also be formulated as a liquid additive packaged in a small container with a dispenser such as a dropper/pipette so that the user can place a drop of the liquid additive in/on the beverage or food, respectively.

An indicator substance configured to create an identifiable or bitter taste when in contact with target substance may be delivered to the user in any suitable form, such as through a pill, a readily-dissolvable tablet, or through a liquid solution. The indicating substance can also be, or be impregnated in, any chewable or edible item that resembles a common beverage or food item, such as a stick or piece of gum, breath mint, mint, candy, such as those candies sold under the federally registered trademark TIC TAC, etc. The substance can therefore be used openly in a manner that would not otherwise alert others that the user is performing a test for the presence of unwanted drugs in a beverage or food item. Alternatively, the substance may be added to the beverage or food itself to produce an identifiable and/or bitter taste.

In some embodiments, the test may include a user placing a detector substance in his or her mouth, then drinking the beverage or eating the food in question. The detector substance may cause the user to have a pre-selected indicator taste. For example, a user may place a dissolvable strip of material in their mouth to moisten and/or dissolve the strip, thereby releasing the substance. The substance may then coat all or a portion of the interior of the user's mouth, including the taste buds, with the substance, so that the test may be performed by subsequently sipping, ingesting, or tasting a small portion of the suspected beverage or food. The results of the test are therefore fully concealed from others and only known to the taster.

All of the compositions, substances and methods disclosed herein can be made and executed without undue experimentation in light of the present disclosure. While the compositions, substances and methods of this disclosure have been described in terms of preferred embodiments, it will be apparent to those of skill in the art that variations may be applied to the compositions/substances, and in the steps or in the sequence of steps of the method described herein without departing from the concept, spirit and scope of the present disclosure. More specifically, it will be apparent that certain substances that are both chemically and physiologically related may be substituted for the substances described herein while the same or similar results would be achieved. All such similar substitutes and modifications apparent to those skilled in the art are deemed to be within the spirit, scope and concept of the present disclosure.

In an alternative embodiment, the non-visual verification system may correspond to a device that incorporates a sensing component and an indicating component. In this embodiment, the sensing component may incorporate a sample chamber or receptacle, where an aliquot of the ingestible substance to be tested may be inserted for sensing. Alternatively, the sensing component may be configured to be inserted into the ingestible substance, such as a dipstick, test strip, or capillary tube. In one embodiment, the sensing component may include a removable sample collector that is configured to be removed from the non-visual verification system and used to collect an aliquot of the ingestible substance of interest. The sample collector may then be reattached or reinserted into the non-visual verification system for analysis. Alternatively, the non-visual verification system may incorporate one or more stages of sample treatment, such as by the addition of water or other solvent, one or more reagents, and optionally including maceration or mixing in order to facilitate detection of a target substance.

The sensing component may incorporate any of a variety of chemical sensors, electrochemical sensors, spectrometric sensors, pH sensors, and the like. Highly selective detection may be accomplished via the use of enzyme-linked-bound antibodies that bind specifically to selected target substances, and then generate a detectable response via an enzymatic reaction (for example as used in ELISA assays). The sensing component may include only one type of chemical sensor, or may incorporate a plurality of chemical sensors to permit detection of more than one target substance.

Alternatively, the sensing component may utilize one or more microfluidic systems to analyze an ingestible substance for the target substance. Microfluidics permit the manipulation of extremely small volumes of analyte utilizing microfluidic channels having diameters on the order of around 100 nm to several hundred pm. Microfluidic methods include a wide variety of analytical methods, leading to the description of microfluidic methods as ‘labs-on-a-chip’.

Where the non-visual verification system incorporates distinct sensing and indicating components, the non-visual verification system may also incorporate a processor 30, as shown in FIG. 3. The processor may correspond to one or more chips, transistors, and processing units configured to receive data originating from the sensing component, evaluating the sensing data, and triggering the indicator component to create the appropriate non-visual indication.

Where the indicating component incorporates a physical indicator that responds to instructions from the processor, the indicating component may generate any of a variety of possible non-visual indications, including smell, touch and sound. For example, the indicating component may be configured to release an appropriate fragrance upon detection of a particular target substance. Alternatively, the indicating component may include a tactile indicator portion, either affixed to the non-visual verification system, or configured to be used remotely. The tactile indicator may be configured to alert the user by vibrating, administering a gentle electrical shock to the users skin, or applying localized heating, for example. When used remotely, the tactile indicator may take the form of a patch or adhesive button that can be removably affixed to the users skin, for example under clothing, and then activated by the non-visual verification system. Remote communication between the non-visual verification system and the tactile indicator may be accomplished using radio transmissions, among other methods. A sound indicator could also be used that responds to, for example, a chemical reaction by emitting a sound to indicate that the target substance is present in the ingestible substance.

Although the present invention has been shown and described with reference to the foregoing operational principles and preferred embodiments, it will be apparent to those skilled in the art that various changes in form and detail may be made without departing from the spirit and scope of the invention. The present invention is intended to embrace all such alternatives, modifications and variances that fall within the scope of the appended claims.

What is claimed is:

1. A non-visual verification method, comprising:
selecting an ingestible substance;
using a detector on the selected, ingestible substance;
non-visually indicating whether a target substance is present in the ingestible substance.

2. The method of claim 1, wherein the indicating step is non-visually and concealably indicating whether a target substance is present in the ingestible substance.

3. The method of claim 1, wherein the using step involves using a taste detector.

4. The method of claim 2, wherein the using step involves using a taste detector.

5. The method of claim 1, wherein the using step involves using a touch detector.

6. The method of claim 2, wherein the using step involves using a touch detector.

7. The method of claim 1, wherein the using step involves using a smell detector.

8. The method of claim 1, wherein the using step involves using a sound detector.

9. A non-visual verification method, comprising:
selecting an ingestible substance;
making a detector for non-visual display of whether a target substance is present in the selected, ingestible substance;
operating the detector and non-visually indicating whether a target substance is present in the ingestible substance.

10. The method of claim 9, wherein the making step involves making a detector for non-visual and concealed display of whether a target substance is present in the selected, ingestible substance.

11. The method of claim 10, wherein the operating and indicating step involves operating the detector, and non-visually and concealably indicating whether a target substance is present in the ingestible substance.

12. The method of claim 9, wherein the making step involves making a taste detector.

13. The method of claim 11, wherein the making step involves making a taste detector.

14. The method of claim 9, wherein the making step involves making a touch detector.

15. The method of claim 11, wherein the making step involves making a touch detector.

16. The method of claim 9, wherein the making step involves making a smell detector.

17. The method of claim 9, wherein the making step involves making a sound detector.

18. A non-visual verification system, comprising:
a non-visual, sensing mechanism constructed to verify whether a target drug is present in an ingestible substance by producing a non-visual indicator that can be understood by a user.

19. The system of claim 18, wherein the sensing mechanism is also constructed to be concealable so that the user can limit observation of the indicator by others.

20. The system of claim 18, wherein the sensing mechanism is constructed to produce a taste indicator to the user.

21. The system of claim 19, wherein the sensing mechanism is constructed to produce a taste indicator to the user.

22. The system of claim 18, wherein the sensing mechanism is constructed to produce a touch indicator to the user.

23. The system of claim 19, wherein the sensing mechanism is constructed to produce a touch indicator to the user.

24. The system of claim 18, wherein the sensing mechanism is constructed to produce a smell indicator to the user.

25. The system of claim 19, wherein the sensing mechanism is constructed to produce of sound indicator to the user.

26. A non-visual verification device, comprising:
a non-visual, sensing component constructed to verify whether a target drug is present in an ingestible substance by producing a non-visual indicator that can be understood by a user.

27. The device of claim 26, wherein the sensing component is also constructed to be concealable so that the user can limit observation of the indicator by others.

28. The device of claim 26, wherein the sensing component is constructed to produce a taste indicator to the user.

29. The device of claim 27, wherein the sensing component is constructed to produce a taste indicator to the user.

30. The device of claim 26, wherein the sensing component is constructed to produce a touch indicator to the user.

31. The device of claim 27, wherein the sensing component is constructed to produce a touch indicator to the user.

32. The device of claim 26, wherein the sensing component is constructed to produce a smell indicator to the user.

33. The device of claim 27, wherein the sensing component is constructed to produce a sound indicator to the user.
